(19) United States
(12) Patent Application Publication
(10) Pub. No.: US 2023/0257751 A1
(43) Pub. Date: Aug. 17, 2023

(54) METHODS FOR PROPHYLACTIC AND THERAPEUTIC TREATMENT OF 2019-nCoV USING siRNAs AGAINST TGFB1 AND COX2

(71) Applicant: Sirnaomics, Inc., Germantown, MD (US)

(72) Inventors: David M. EVANS, Germantown, MD (US); Wookhyun KIM, Germantown, MD (US)

(21) Appl. No.: 18/111,309

(22) Filed: Feb. 17, 2023

Related U.S. Application Data
(63) Continuation of application No. PCT/US22/49406, filed on Nov. 9, 2022.
(60) Provisional application No. 63/277,576, filed on Nov. 9, 2021.

(51) Int. Cl. C12N 15/113; A61P 31/14

(52) U.S. Cl. CPC C12N 15/1136; C12N 15/1137; A61P 31/14; C12N 2310/11

(57) ABSTRACT

Methods are provided for prevention and treatment of 2019 coronavirus (2019-nCoV; COVID-19) infections in mammals by prophylactic or therapeutic administration of pharmaceutical compositions known as STP707, which compositions have been previously disclosed. These compositions comprise potent siRNA therapeutics formulated in a histidine-lysine polymeric carrier; the siRNA molecules target and reduce or inhibit TGFβ1 and Cox2 gene expression, preventing or ameliorating COVID-19 symptoms.

### CROSS REFERENCE TO RELATED APPLICATIONS

This application is a continuation of international application PCT/US2022/049406, filed Nov. 9, 2022, which claims the benefit of and priority to U.S. Provisional Patent Application No. 63/277,576, filed Nov. 9, 2021, the contents of which are incorporated herein by reference in their entirety.

### SEQUENCE LISTING

The instant application contains a sequence listing, submitted electronically in ST.26 (XML) format, which is hereby incorporated by reference in its entirety. The ST.26 sequence listing, created on Nov. 6, 2022, is named 4690.0060i sequences xml ST.26 and is 72 kilobytes in size.

### FIELD

Methods are provided for administration of nucleic acid-containing polymeric nanoparticles to prevent and treat 2019-nCoV infections and the resulting respiratory disease, COVID-19, in mammals. Administration of RNA-containing histidine-lysine polymeric nanoparticles modulate, interfere with, and/or inhibit TGFβ1 and Cox2 gene expression.

### BACKGROUND

2019-nCoV Infection/COVID-19: Biology and Pathology

On Dec. 31, 2019, the World Health Organization (WHO) China Country Office was informed of cases of pneumonia of unknown etiology detected in Wuhan City, Hubei Province of China. The WHO reported that a novel coronavirus (2019-nCoV), a single-stranded, positive-sense RNA betacoronavirus (βCoV), was identified as the causative virus by Chinese authorities on 7 January (Lu H, Stratton C W, Tang Y W. Outbreak of Pneumonia of Unknown Etiology in Wuhan China: the Mystery and the Miracle. J Med Virol. 2020 Jan. 16.). The 2019-nCoV is highly transmissible and the resulting respiratory disease, COVID19, has been fatal to large numbers of infected individuals. To-date over 250 million cases have been verified worldwide, and over 5 million people have died.

Symptoms of 2019-nCoV infection are similar to a range of other illnesses such as influenza, and include, among others, fever, coughing and difficulty breathing, the latter, which indicates the need for immediate medical attention. Clinical manifestations subsequent to infection include severe pneumonia, acute respiratory distress syndrome, septic shock and multi-organ failure. The 2019-nCoV infection may appear clinically milder than Severe Acute Respiratory Syndrome (SARS) or Middle East Respiratory Syndrome (MERS) in terms of fatalities and transmissibility, although new variants emerging from “super-spreader” events may challenge this observation.

The 2019-nCoV is most closely related to two bat SARS-like coronavirus samples from China, initially suggesting that, like SARS and MERS, it may have originated in bats. (Ji W, Wang W, Zhao X, Zai J, Li X). Genomes and sub-genomes of coronaviruses (CoVs) contain at least 6 open reading frames (ZORFs). The first ORF (ORF1a/b), about two-third of genome length, encodes 16 non-structural proteins (nsp1-16), except Gamma coronavirus that lacks nsp1. There is a −1 frameshift between ORF1a and ORF1b, leading to production of two polypeptides: pp1a and pp1ab. These polypeptides are processed by virally encoded chymotrypsin-like protease (3CLpro) or main protease (Mpro), and one or two papain-like protease (PLPs) into 16 nsps (Ziebuhr J, Snij der E J, Gorbalenya A E. Virus-encoded proteinases and proteolytic processing in the Nidovirales. J Gen Virol. 2000; 81(Pt 4): 853-879.). Other ORFs on the one-third of genome near the 3′ terminus encode at least four main structural proteins: spike (S), membrane (M), envelope (E), and nucleocapsid (N) proteins.

A 2019-nCoV was isolated from a patient in January 2020 and subjected to genome sequencing, showing that the 2019-nCoV is a βCoV of group 2B with at least 70 percent similarity in genetic sequence to SARS-CoV. Sequence analysis showed that the 2019-nCoV possesses a typical genome structure of a CoV and belongs to the cluster of βCoVs that include Bat-SARS-like (SL)-ZC45, Bat-SL ZXC21, and SARS-CoV.

### TGFβ1 and Cox2

Transforming growth factor beta 1 (TGFβ1) and cyclooxygenase 2 (Cox2) or prostaglandin-endoperoxide synthase 2 (PTGS2) have each been implicated in disease progression, including skin cancers and other conditions. Previously it has been shown that administration of two siRNAs targeting TGF-β1 and Cox2 in a single nanoparticle formulation permits entry of the siRNAs into the same cells at the same time, and that silencing of target genes in these cells results in antitumoral activity (in patients with in situ squamous cell carcinoma (isSCC)), improved wound healing and the resolution of hypertrophic scars. The family of TGFβs are involved in a variety of cellular functions, including immune system suppression and can be over-expressed in a number diseases and disordered conditions, including cancer, elevated inflammation as well as in early stages of severe COVID-19.

### RNAi

RNA interference (RNAi) is a sequence-specific RNA degradation process that provides a direct way to knockdown, or silence, theoretically any gene. In naturally occurring RNA interference, a double stranded RNA is cleaved by an endonuclease into small interfering RNA (siRNA) molecules, overhangs at the 3′ ends. These siRNA molecules are incorporated into a multicomponent-ribonuclease called RNA-induced-silencing-complex (RISC). One strand of siRNA remains associated with RISC and guides the complex towards a cognate RNA that has sequence complementary to the guider single stranded siRNA (ss-siRNA) in RISC. This siRNA-directed endonuclease digests the RNA, thereby inactivating it. Studies have revealed that the use of chemically synthesized 21-25-nucleotide (nt) siRNA molecules exhibit RNAi effects in mammalian cells, and the thermodynamic stability of siRNA hybridization (at or between terminals) plays a central role in determining the molecule's function.

### SUMMARY

Methods are provided for preventing and treating 2019-nCoV infections in mammals using IV or IT (intratracheal) administration of nucleic acid-containing pharmaceutical compositions. The compositions (described in detail, e.g., in U.S. Pat. Nos. RE46,873 and 9,642,952) comprise nucleic acids (e.g., siRNA, mRNA, and miRNA) and histidine-lysine copolymers carrier such as HKP or HKP(+H). When formulated, the components self-assemble as nanoparticles that, once inside the target cells, effectively modulate, interfere with, and/or inhibit TGFβ1 and Cox2 gene expression. The compositions comprising siRNA molecules targeting the expression of TGFβ1 and Cox2 genes are Sirnaomics' STP707 products. Sirnaomics' STP705 products comprise the same combination of siRNAs.

In certain embodiments methods are provided for preventing the 2019-nCoV infection by prophylactically administering a pharmaceutically effective amount of STP707 to a subject in need. In certain embodiments, administration may take place prior to exposure of the subject to 2019-nCoV. In a variety of embodiments, administration can take place at up to 2 weeks in advance to within 3 to 24 hours of exposure to the virus.

In other embodiments methods are provided for treating a subject with STP707 administration who has, or is suspected of having, been infected with 2019-nCoV. In some embodiments, the subject has not exhibited any of the known symptoms of the infection. In a number of embodiments, STP707 is administered at each of a number of periods following the subject's exposure to the virus: within at least 3 hours and up to 8 weeks following any suspected or known exposure to the virus.

In certain embodiments the subject exhibits elevated serum TGFβ1 levels as compared to those in a standard range applicable to healthy subjects. In other embodiments a hospitalized subject diagnosed with a 2019-nCoV infection is treated with therapeutic administration of STP707 after exhibiting elevated serum TGFβ1 levels anytime between the day of admittance to the hospital and to up to 21 days following admittance; the subject is administered the STP707 initially within 1 to 4 days following initial detection of said elevated TFGβ1 levels.

In some embodiments methods are provided for slowing the progression of a 2019-nCoV infection in a subject, comprising administering to the subject a pharmaceutically effective amount of STP707. In certain embodiments, STP707 is administered within 3 to ˜24 hours and up to 8 weeks following exposure of the subject to the virus.

In all embodiments, the compositions may be administered through a variety of routes, including, but not limited to, intratracheal (IT), intranasal (inhaled), and intravenous (IV) injection or infusion. The subject is a mammal, in particular, a human, or alternatively a non-human primate, rat, mouse, ferret, or other mammal.

### BRIEF DESCRIPTION OF THE DRAWINGS

FIG. 1 depicts the PNP platform for delivering siRNA molecules, together with histidine-lysine copolymer in the same nanoparticle.

FIG. 2 shows the effect of IV administration of STP707 on TGFβ1 and Cox2 gene expression in liver and lung tissue of rhesus macaques infected with 2019-nCoV.

FIG. 3 shows a schematic of the study design for evaluating the efficacy of prophylactic or therapeutic STP707 administration in mice infected with 2019-nCoV. The study is described in Example 2 below.

FIG. 4 shows the change (loss) of body weight due to infection, and, for the STP707 prophylactically treated groups, recovery from the infection. Mice received one dose of STP707 prophylactically through intratracheal (IT) or intravenous (IV) administration routes, and three doses following the date of 2019-nCoV infection.

FIG. 5 (A-D) show the change in body weight progression for individual mice in the control (A-B) and treatment groups (C-D). Two of 6 mice that received STP707 prophylactically through intratracheal (IT) administration maintained and gained weight, while the other four continued to lose weight and either died or were euthanized at Day 7 or 8 due to extreme weight loss. For the mice administered STP707 intravenously (IV), all five animals initially lost weight as the infection progressed in the first week but recovered through the second week of the study.

FIG. 6 shows the change (loss) of body weight due to infection, and, for only the group of mice that received intravenous (IV) therapeutic administration of STP707 groups, recovery from the infection through the second week of the study. Mice received therapeutic intratracheal (IT) or intravenous (IV) administration of STP707 (one dose) on the date of and (three doses) after infection with 2019-nCoV, but only IV administration of STP707 was efficacious.

FIG. 7 (A-D) show the change in body weight progression for individual mice in the control (A-B) and treatment groups (C-D). All 6 mice that received STP707 after infection with 2019-nCoV through intratracheal (IT) administration had died or were euthanized due to extreme weight loss by Day 7. For the mice administered STP707 intravenously (IV) following infection, only one recovered in the second week following infection.

FIG. 8 shows mortality in both the prophylactic and therapeutic STP707 regimens. All mice in the control groups had died by Day 7 post infection, but prophylactic delivery of STP707 via the intravenous route resulted in an 83 percent survival rate by Day 14.

FIG. 9 (Table 1) shows the gene-specific primers and probe for amplification and quantification of TGFβ1 in extracted RNA.

FIG. 10 (Table 2) shows gene-specific primers and probe for amplification and quantification of GAPDH (IDT DNA Mm.PT39a.1).

FIG. 11 (Table 3) shows data and calculated values for TGFβ1 gene expression in mouse lung tissue.

FIG. 12 (A-C) show the effect of intratracheal administration of STP707 on TGFβ1 levels in the lung after one or two doses: (A) relative TGFβ, DDCt GAPDH; (B) TGFβ, normalized to RNA; and (C) GAPDH, normalized to RNA. A dose-dependent reduction in TGFβ1 is seen when STP707 is administered intratracheally in mice, suggesting that intratracheal administration of STP707 targeting TGFβ1 gene expression is capable of penetrating and silencing targeted genes in mouse lung tissue.

### DETAILED DESCRIPTION

Methods are provided for the prevention and treatment of 2019-nCoV infections by administering a pharmaceutical composition comprising two small interfering RNA (siRNA) molecules formulated with a histidine-lysine copolymer into nanoparticles. The two siRNAs packaged into each nanoparticle, when delivered to a target cell, are internalized and inhibit or reduce the expression of at least two genes of interest, TGFβ1 and Cox2, and thereby prevent or ameliorate the symptoms of a 2019-nCoV infection (and, hence, the respiratory disease, COVID-19) in mammals. The pharmaceutical compositions for STP707 contain siRNAs that target TGFβ1 and Cox2, together with the histidine-lysine copolymer carrier, and have previously been disclosed in detail in U.S. Pat. No. 9,642,873 (873 patent). The STP707 and STP705 pharmaceutical compositions and their preparation are described below. The STP705 and STP707 compositions contain the same two siRNA molecules targeting TGFβ1 and Cox2 genes.

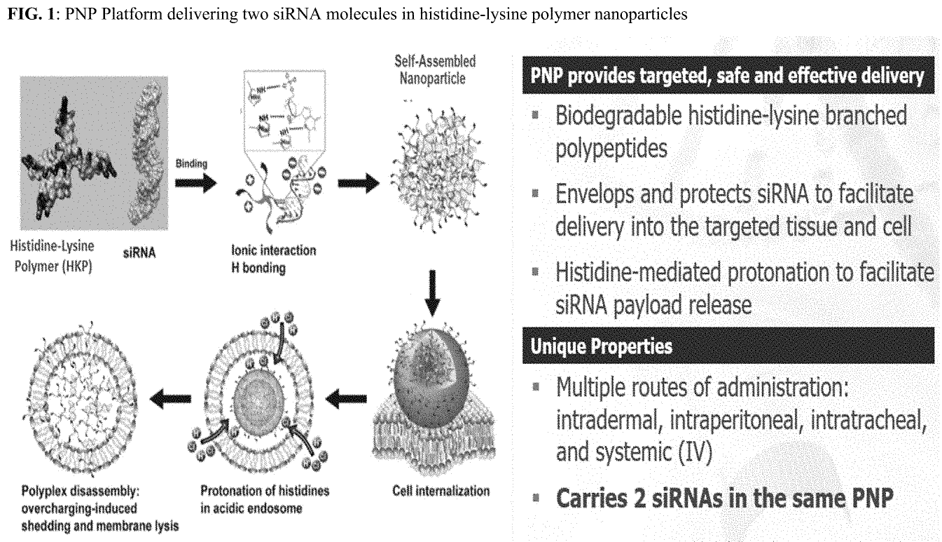

The PNP platform for delivering one or more nucleic acids (siRNA, miRNA, mRNA) molecules to targeted tissues and cells is shown in FIG. 1 and has been described previously. Briefly, products such as STP707 are formulated as pharmaceutical compositions containing nucleic acids (here, two siRNAs targeting the TGFβ1 and Cox2 genes, respectively) and histidine-lysine copolymers, which, when mixed as aqueous solutions in a 2.5:1 to 4:1 (HKP:siRNA) ratio, spontaneously form nanoparticles with an average diameter of 100-150 nm, capable of being internalized in targeted cells following local or systemic administration by any number of routes. The particles disassemble once inside the cells, releasing the nucleic acids to exert their respective effects. Further detail on histidine-lysine copolymer carriers has been discussed previously in the '873 patent.

TGFβ1 and Cox2 and their Measurement

TGFβ1 is the predominant isoform of the TGFβs in the immune system, promoting certain inflammatory actions and suppressing others, and is a primary target for inhibitors in a variety of diseases and disorders.

COX-2 is involved in diseases associated with dysregulated inflammatory conditions, such as rheumatoid and osteoarthritis, cardiovascular disease, and the carcinogenesis process. COX-2 undergoes immediate-early up-regulation in response to an inflammatory stimulus, and functions by producing prostaglandins that control many aspects of the resulting inflammation, including the induction of vascular permeability and the infiltration and activation of inflammatory cells.

An immunohistochemistry assay may be used to measure the protein expression levels of both TGFβ1 and Cox2. Thus, paraffin embedded sections are deparaffinized in xylene and rehydrated through a graded series from ethanol to water. Antigen retrieval may be performed by heating slides immersed in EDTA-Tris solution (10 mM Tris, 1 mM EDTA, pH9) using a microwave oven for 15 min at low temperature. After washing sections with PBS, endogenous peroxidase activity is blocked by incubation in 3% hydrogen peroxide for 10 min. and 5% bovine serum albumin (BSA) for 20 min. The sections are incubated overnight at 4° C. with rabbit polyclonal antibodies (anti-Cox2), using PBS as the negative control. The sections are washed with PBS and then incubated with a biotinylated goat anti-rabbit antibody for 20 min at room temperature, followed by another wash with PBS. The sections are incubated with SABC for 20 min. at room temperature, followed by washing with PBS. Freshly prepared 3,3-diaminobenzidine tetrahydrochloride is used to visualize antibody binding. Sections are counterstained with hematoxylin, followed by dehydration; clearing and overslipping. The TGFβ1 protein staining was significantly down-regulated after in vitro transfection of STP705 to the organ culture. Cox-2 protein staining also was significantly down-regulated after in vitro transfection of STP705 to the organ culture.

Both TGFβ1 and Cox2 in tissue also can be measured using real time quantitative reverse transcription PCR (qRT-PCR). In serum they can be quantified using an enzyme-linked immunosorbent assay (ELISA) using a commercially available kit, e.g., those available through Invitrogen or Novus Biologicals (TGFβ1), and RayBiotech and Enzo Life Sciences (Cox2).

### RNAi

RNA interference (RNAi) is a naturally occurring, highly specific mode of gene regulation. The mechanics of RNAi are both exquisite and highly discriminating. At the onset, short (19-25 bp) double-stranded RNA sequences (referred to as short interfering RNAs, siRNAs) associate with the cytoplasmically localized RNA Interference Silencing Complex (RISC). The resultant complex then searches messenger RNAs (mRNAs) for complementary sequences, i.e., target genes or sections thereof, eventually degrading (and/or attenuating translation of) these transcripts. Scientists have co-opted the endogenous RNAi machinery to advance a wide range of uses for siRNAs, including as therapeutics.

The nucleic acid may be a small interfering RNA (siRNA) molecule, comprising a double stranded (duplex) oligonucleotide, wherein the oligonucleotide targets a complementary nucleotide sequence in a single stranded (ss) target RNA molecule. The ss target RNA target molecule is an mRNA encoding at least part of a peptide or protein whose activity promotes inflammation, adipose tissue remodeling or sculpting, wound healing, or scar formation in skin tissue, or it is a micro RNA (miRNA) functioning as a regulatory molecule. siRNA sequences may be prepared in such way that each duplex can target and inhibit the same gene from, at least, both human and mouse, or non-human primates. In certain embodiments, an siRNA molecule binds to an mRNA molecule that encodes at least one protein with 100 percent or less complementarity. In further embodiments, an siRNA molecule binds to a mRNA molecule that encodes at least one human protein. In still additional embodiments, an siRNA molecule binds to a human mRNA molecule and to a homologous mouse mRNA molecule, i.e., mRNAs in the respective species that encode the same or similar protein.

“RNA” refers to a molecule comprising at least one, and preferably at least 4, 8 and 12 ribonucleotide residues. The at least 4, 8 or 12 RNA residues may be contiguous. By “ribonucleotide” is meant a nucleotide with a hydroxyl group at the 2′ position of a β-D-ribofuranose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the dsRNA or internally, for example at one or more nucleotides of the RNA. Nucleotides in the RNA molecules of the disclosed embodiments can also comprise non-standard nucleotides, such as non-naturally occurring nucleotides or chemically synthesized nucleotides or deoxynucleotides. These altered RNAs can be referred to as analogs or analogs of naturally occurring RNA. As used herein, the term “siRNA” refers to a double stranded nucleic acid in which each strand comprises RNA, RNA analog(s) or RNA and DNA. Typically, the antisense strand of the siRNA is sufficiently complementary with the identified target sequences.

Prevention of and Treatment for 2019-nCoV Infection/COVID-19 and Other Viral Infections

Prophylactic and therapeutic methods are provided for preventing or treating a 2019-nCoV infection (and COVID-19) in a subject who may or may not have been exposed to the virus or who has been diagnosed with the infection, with the goals of ameliorating symptoms, slowing the progression of the disease and, ultimately, of reducing the possibility of the subject's death in the most severe COVID-19 cases. Methods for preventing or treating such a viral infection comprise administering STP707 through IV, IT, or other routes, as discussed below. STP707 may provide significant value as a prophylactic/therapeutic with broad virus strain coverage and this coverage may well extend to as yet unidentified strains that may emerge in the future.

“Treatment,” or “treating” as used herein, is defined generally as the application or administration of a therapeutic agent (e.g., a dsRNA agent or vector or transgene encoding same) to a subject, or application or administration of a therapeutic agent to an isolated tissue or cell line from a subject, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

One aspect of the disclosed embodiments relates to treating subjects prophylactically, i.e., either to prevent the onset of symptoms of an infection, or to lessen the severity of symptoms if a 2019-nCoV infection should occur. In either case, the subject may not know whether s/he has been exposed to the virus at the time of STP707 administration. In other embodiments, the subject may know that exposure took place.

Another aspect of the disclosed embodiments pertains to methods of treating subjects therapeutically, i.e., altering the onset or the anticipated onset of symptoms of the 2019-nCoV infection.

In other aspects, methods of treatment using STP707 may include combining it with one or more other treatments to prevent and treat a 2019-nCoV infection, including addressing amelioration of particular symptoms. In certain of these embodiments, the other treatments involve administration of other nucleic acids (other siRNA, miRNA, mRNA, etc.) to comprise a treatment regimen comprising 3, 4, 5, or up to 10 individual nucleic acids targeting up to 10 or more different genes. In still other embodiments the other treatments involve administration of one or more small molecules or aptamers, antibody-based treatments, etc. In other embodiments, STP707 and other treatment(s) combinations may be administered to prevent or treat other viral infections as well as for 2019-nCoV infection or any of its variants. Some embodiments may involve administration of STP707 and the one or more other treatments simultaneously. Other method embodiments may involve staggered administration of STP707 and the other treatment(s), and further may involve prophylactic as well as therapeutic administration of STP707 and the same for any of the other treatments if such indications are approved.

Other method embodiments for preventing a viral infection such as 2019-nCoV or its variants comprise administering STP707 over periods varying in length from weeks to months prior to and/or following exposure. In still other method embodiments, administration of STP707 at full or partial doses at regular or irregular intervals prior to or following dosing with other treatments may avoid or lessen the severity of symptoms of a viral infection, including 2019-nCoV and its variants.

It has recently been reported that serum TGFβ1 is significantly elevated in a subset of newly diagnosed subjects with moderate, and especially, severe COVID-19 within their first week of hospitalization, while another set of less severely affected, hospitalized 2019-nCoV subjects exhibited a later rise in TGFβ1 (beginning in the third week of hospitalization). This early rise in TGFβ1 in subjects with more severe symptoms could be responsible for inhibiting of the normal, rapid rise of NK cells in response to the infection in these very ill subjects. In those subjects without such severe symptoms the rapid rise of NK population suppresses the rise in TGFβ1 until at least the third week following admittance (Witkowski et al. Nature. https://doi.org/10.1038/s41586-021-04142-6 (2021); Chen. A potential treatment of COVID-19 with TGF-β blockade. Int'l J. Biol. Sci. 16(11):1954-1955, 2020, doi:10.7150/ijbs.46891). For subjects hospitalized with COVID-19, and especially those with moderately severe or severe symptoms, methods include testing for serum TGFβ1 immediately upon admittance or during their first week in the hospital; if TGFβ1 is elevated in the first few days in a subject severely affected, the administration of STP707 can reduce its expression, permitting the NK cell response normally present in response to an infection such as 2019-nCoV.

### STP707 Efficacy in Cynomolgus Monkeys

The ability for STP707 to reduce expression of TGFβ1 and Cox2 in the liver and lung tissue of Cynomolgus monkeys was evaluated after IV administration of STP707 at 4 mg/kg/week for four weeks or 6 mg/kg/week for 6 weeks. FIG. 3 shows the reduction of TGFβ1 and Cox2 expression in each tissue following IV administration of STP707 in the 4- and 6-week cohorts. The effect was apparently more evident in liver, but there was a highly significant reduction in expression of TGFβ1 in lung in the as well at four weeks (versus 6), indicating that IV administration resulted in delivering at least the siRNA against TGFβ1 to the lung. Further, for both TGFβ1 and Cox2, expression in the lung appears to trend downward. See further detail in Example 1 below.

### Timing and Mode of Administration in Mice

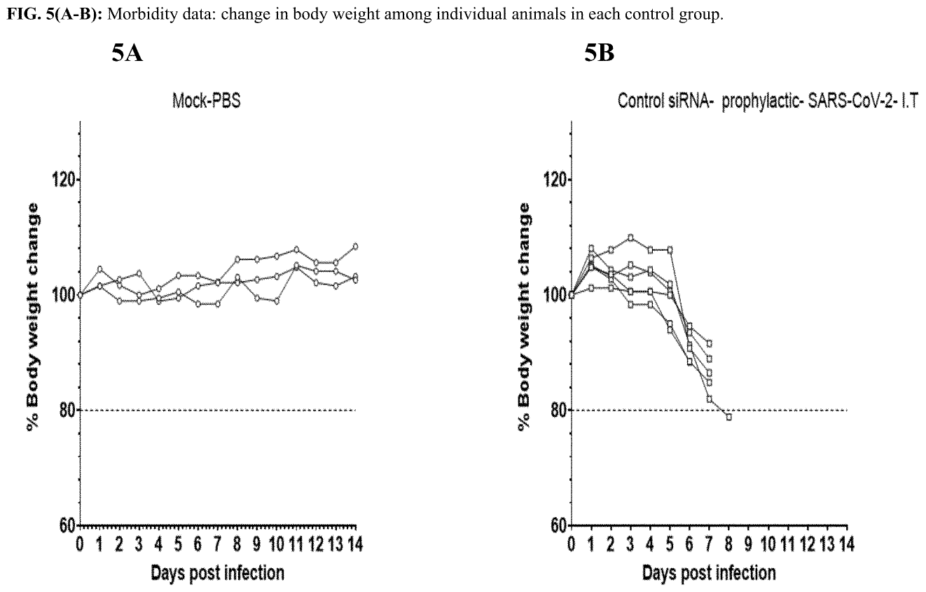

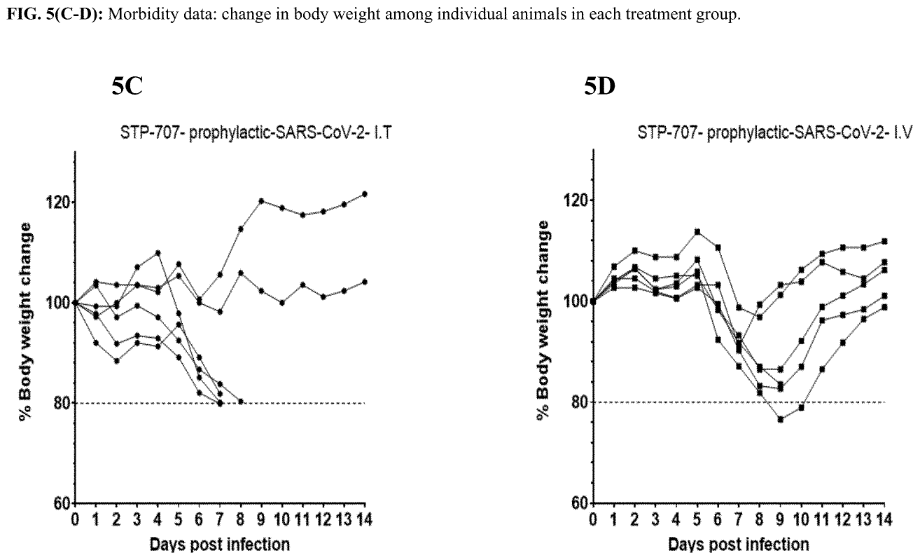

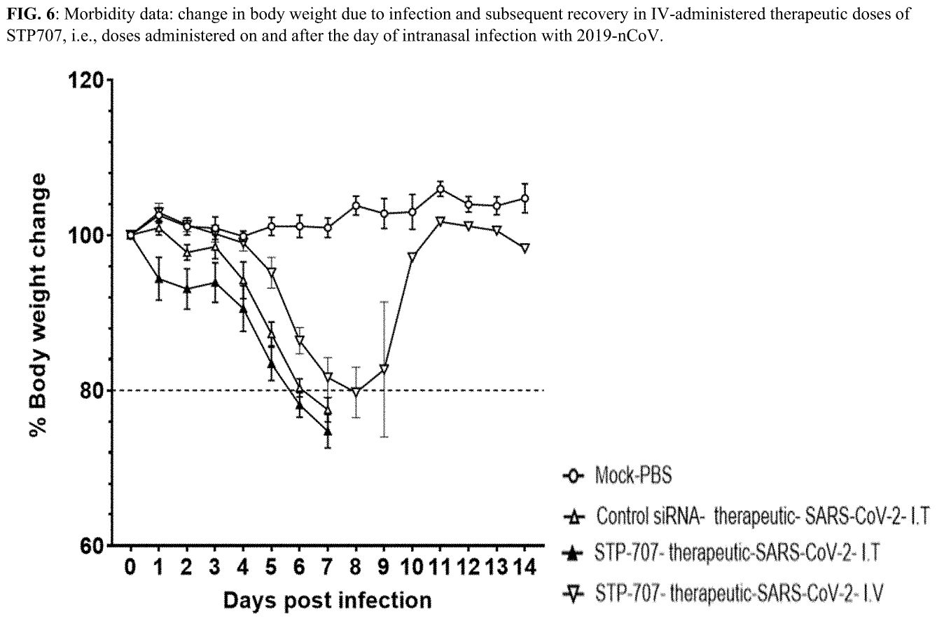

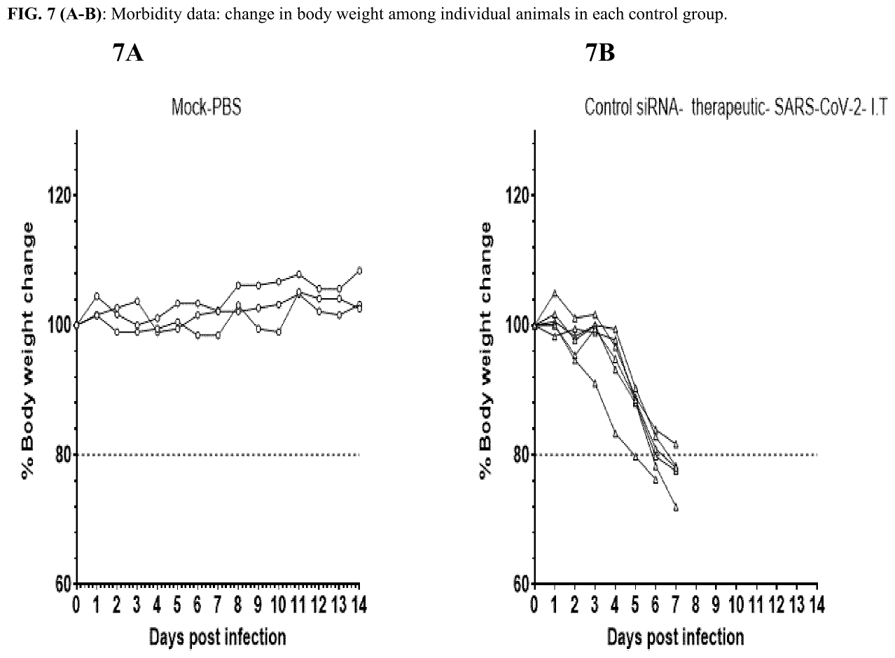

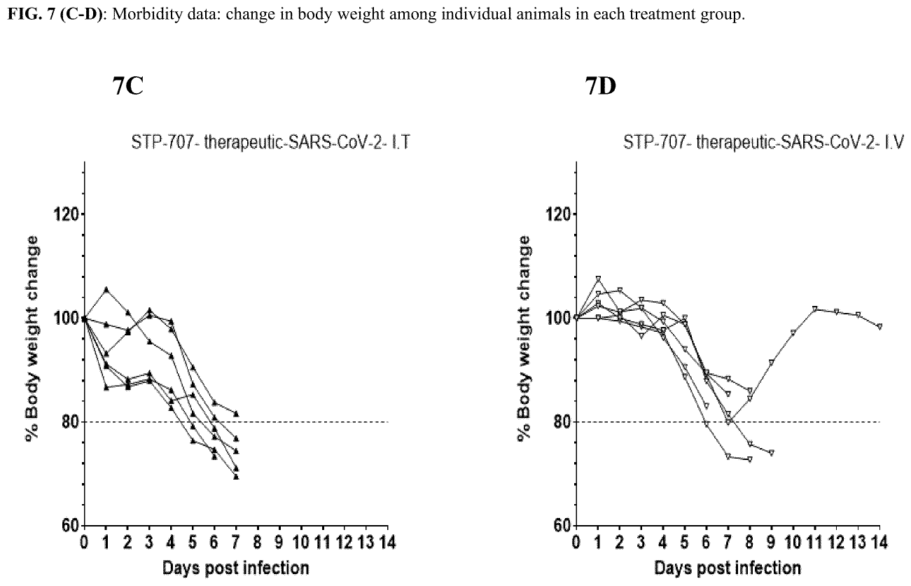

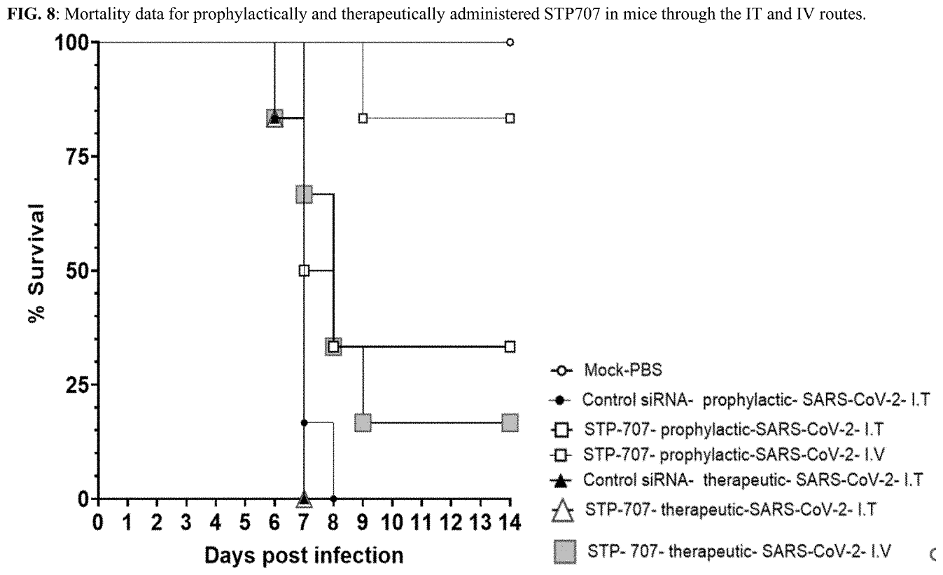

FIG. 5 (prophylactic administration, IT and IV) and FIG. 7 (therapeutic administration, IT and IV) show the rapid reduction in body weight within a few days after infection. The data show equally rapid recovery of body weight in both treatment groups (IT and IV) when STP707 was administered prophylactically (FIG. 5), but when the composition was administered on the day (and following) infection, only the IV route of administration was effective (FIG. 7). All mice in the IT-administered treatment group died or had to be euthanized by Day 7. FIGS. 6A-6D and 8A-8D, depict the change of body weight post infection among individual mice in the four (2 prophylactic, 2 therapeutic) groups, showing a stronger response to administration of STP707 among mice treated prophylactically. FIG. 8D in particular, however, reveals that only one therapeutically treated mouse rebounded after initial weight loss survived to live to Day 14, while all others in that IV-administered treatment group had died or were euthanized due to extreme weight loss. See further detail in Example 2 below.

### STP707 Efficacy in CD-1 Mice

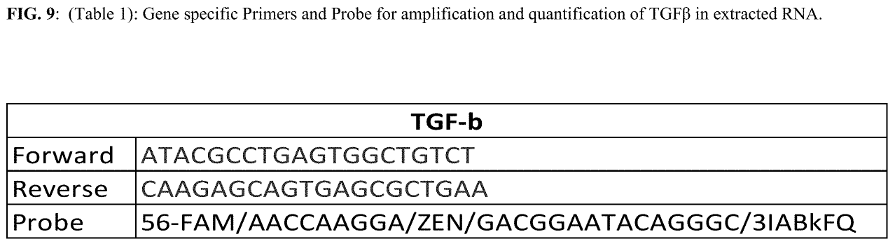

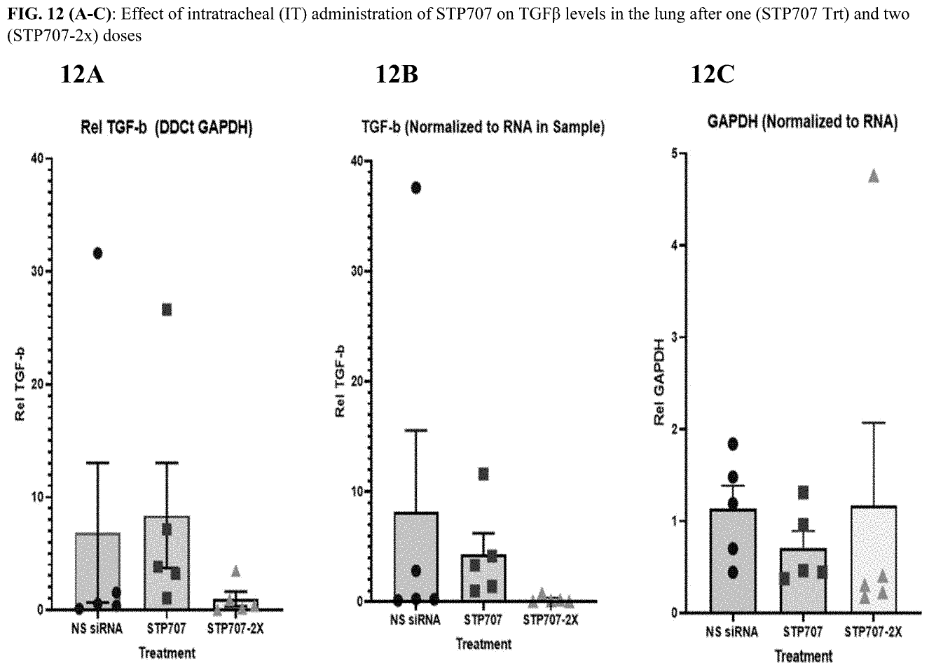

A three-day study in mice to evaluate the efficacy of STP707 was carried out as further described in Example 3. FIGS. 9-11 provide tables showing primers and probes for the genes of interest, and the resulting TGFβ1 gene expression in mouse lung tissue. FIG. 12 (A-C) show the effect of intratracheal administration of STP707 on TGFβ1 levels in the lung after one or two doses. A dose-dependent reduction in TGFβ1 was seen when STP707 was administered intratracheally (IT) in mice in one or two doses, suggesting that IT administration of the histidine-lysine copolymer along with the siRNA molecules targeting TGFβ1 and Cox2 genes is capable of penetrating mouse lung tissue and silencing the targeted genes, indicating that HKP delivered the two siRNAs through the IT route.

Prophylactic and therapeutic methods of treatment may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype,” or “drug response genotype”). Thus, another aspect of the disclosed embodiments provides methods for tailoring a subject's prophylactic or therapeutic treatment with either the target TGF-β1 and Cox2 genes or modulators according to that subject's drug response genotype. Pharmacogenomics allows a clinician or physician to target prophylactic or therapeutic treatments to patients who will most benefit from the treatment and to avoid treatment of patients who will experience toxic drug-related side effects.

### Dosing

STP707 can be formulated to comprise a pharmacologically effective amount of therapeutic agent, i.e., of each of the two siRNA molecules, along with a pharmaceutically acceptable carrier.

A pharmacologically or therapeutically effective amount refers to that amount of the RNA effective to produce the intended pharmacological, therapeutic or preventive result. The phrases “pharmacologically effective amount” and “therapeutically effective amount” or simply “effective amount” refer to that amount of an RNA effective to produce the intended pharmacological, therapeutic or preventive result. For example, if a given clinical treatment is considered effective when there is at least a 20 percent reduction in a measurable parameter associated with a disease or disorder, a therapeutically effective amount of a drug for the treatment of that disease or disorder is the amount necessary to effect at least a 20 percent reduction in that parameter.

As defined herein, a therapeutically effective amount of a nucleic acid molecule (i.e., an effective dosage) depends on the nucleic acid selected. For instance, single dose amounts of an RNA molecule (or, e.g., a construct(s) encoding for such RNA) in the range of approximately 1 pg to up to 10 mg may be administered; in some embodiments, 1, 10, 30, 100, or 1000 pg, or 10, 30, 100, or 1000 ng, or 10, 30, 100, or 1000 μg, may be administered in several areas of the body of a 60 to 120 kg subject (i.e., 0.1 μg/Kg to 2000 μg/Kg). In some embodiments, doses ranging from 60 to 150 μg are administered in this way. The compositions can be administered from one or more times per day to one or more times per week for the desired length of the treatment; including once every other day. The skilled artisan will appreciate that certain factors may influence the dosage and timing required to effectively treat a subject, including but not limited to the severity of the disease or disorder, previous treatments, the general health and/or age of the subject, and other diseases present. Treatment of a subject with a therapeutically effective amount of a nucleic acid (e.g., dsRNA), protein, polypeptide, or antibody can include a single treatment or, preferably, can include a series of treatments.

A pharmaceutical composition comprising the RNA can be administered once daily. However, the therapeutic agent may also be dosed in units containing two, three, four, five, six or more sub-doses administered at appropriate intervals throughout the day. In that case, the RNA contained in each sub-dose must be correspondingly smaller in order to achieve the total daily dosage unit. The dosage unit can also be compounded for a single dose over several days, e.g., using a conventional sustained release formulation which provides sustained and consistent release of the RNA over a several day period. Sustained release formulations are well known in the art. In this embodiment, the dosage unit contains a corresponding multiple of the daily dose. Regardless of the formulation, the pharmaceutical composition must contain RNA in a quantity sufficient to inhibit expression of the target gene in the animal or human being treated. The composition can be compounded in such a way that the sum of the multiple units of RNA together contain a sufficient dose.

Depending on the particular target gene sequence and the dose of RNA agent material delivered, this process may provide partial or complete loss of function for the target gene. A reduction or loss of expression (either target gene expression or encoded polypeptide expression) in at least 50%, 60%, 70%, 80%, 90%, 95% or 99% or more of targeted cells is exemplary. Inhibition of target gene levels or expression refers to the absence (or observable decrease) in the level of target gene or target gene-encoded protein. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (MA), other immunoassays, and fluorescence activated cell analysis (FACS). Inhibition of target gene sequence(s) by the dsRNA agents of the disclosed embodiments also can be measured based upon the effect of administration of such dsRNA agents upon development/progression of a target gene associated disease or disorder, e.g., deleterious adipose tissue remodeling due to obesity, over feeding or a metabolic derangement, tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 105-fold, 106-fold, 107-fold reduction in cancer cell levels could be achieved via administration of the dsRNA agents of the disclosed embodiments to cells, a tissue, or a subject.

The data obtained from the cell culture assays and animal studies (toxicity, therapeutic efficacy) can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in the method of the disclosed embodiments, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography or by an enzyme-linked immunosorbent assay.

### Administration for Prevention and Treatment

Biological barriers protect the lungs from foreign particles. Examples include (i) a thick mucus layer that may bind inhaled drugs and remove them via a mucus clearance mechanism, (ii) low basal and stimulated rates of endocytosis on the apical surfaces of well-differentiated airway epithelial cells, (iii) the presence of RNase extra- and intra-cellularly, and (iv) the presence of endosomal degradation systems in the target cells, among others. Overcoming the difficulties of respiratory tract delivery and effective cellular entry and function will pave the way for siRNA as a systemic anti-2019-nCoV therapeutic.

The delivery vehicle and mode of administration allow a rapid onset of gene silencing at the targeted site of action, e.g., early stage infection/prophylaxis at the epithelial/endothelial cells, and in later stage disease through systemic administration. Among them, topical/systemic delivery through inhalation has been shown an effective way to treat the respiratory system diseases. The data described herein demonstrate that systemic (IV) administration may, in some situations, provide a superior result when IT administration shows lower or even no efficacy. In a pandemic setting, either IT or IV administration may permit ease of administration directly to subjects.

Suitably formulated compositions of the disclosed embodiments can be administered by means known in the art such as by parenteral routes, including intravenous, intramuscular, intraperitoneal, subcutaneous, transdermal, airway (aerosol), rectal, vaginal and topical (including buccal and sublingual) administration. In some embodiments, the pharmaceutical compositions are administered by intravenous or intraparenteral infusion or injection.

COVID-19 is an infectious disease of the respiratory system. It enters the cells through the ACE2 receptors or Neuropilin 1 receptors on the epidermal cells of the respiratory tract and lungs to start the replication life cycle. Therefore, administration through the respiratory tract is a suitable effective mode of administration of STP707 in some situations. The pharmaceutical composition, STP707, is delivered to the respiratory system, especially the lower respiratory tract and lungs through a specific inhalation device, which can effectively reach the focus of virus infection and replication to achieve high-efficiency of the inhibition of virus. One administration mode for the prevention and treatment of novel coronavirus infection is atomized inhalation administration. Specifically, a hand-held atomization device is used to atomize the nanoparticles preparation. The inhaled droplets of the drug preparation are atomized through the respiratory tract to deliver the drugs to the lower respiratory tract and lungs. The device preferably uses an ultrasonic atomization device, and more preferably, a micro-net ultrasonic atomization inhalation device.

A formulation is prepared to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The siRNA formulations can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

Further, the siRNA formulations can also be administered by a method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

2019-nCoV infections or suspected 2019-nCoV infections may be treated to slow progression or infections may be prevented altogether by administering to a subject in need a pharmaceutically effective amount of STP707. STP707 may be administered to a subject initially within 3 hours, 6 hours, 12 hours, 18 hours, 1 day, 2 days, 3 days, 4 days, 5 days, 6 days, 8 days, 10 days, 2 weeks, 3 weeks, 4 weeks, 6 weeks or 8 weeks following initial determination of an elevated TGFβ1 concentration in the subject's blood, or following exposure, suspected exposure or as a preventative measure prior to exposure of the subject to 2019-nCoV, regardless of whether the subject has exhibited any signs or symptoms of a 2019-nCoV infection.

Pharmaceutical Compositions Containing siRNA and Histidine-Lysine Copolymer Carriers, Administered Under the Method Embodiments Disclosed and Claimed Herein

STP707 compositions and methods of making them were disclosed previously in U.S. Pat. No. 9,642,873, containing inhibitors of TGF-β1 and Cox2 gene expression, administered in the method embodiments disclosed and claimed herein.

Additional examples of examples of siRNA molecules against TGFβ1 and Cox2, include:

| SiRNA SEQ (against TGFB1 and Cox2) | SEQ ID No: |
|---|---|
| cccaagggcu accaugccaa cuucu | 1 |
| agaaguuggc augguagccc uuggg | 2 |
| ggucuggugc cuggucugau gaugu | 3 |
| acaucaucag accaggcacc agacc | 4 |
| gguggcugga acagccagau guguu | 5 |
| aacacaucug gcuguuccag ccacc | 6 |
| gaggagccuu caggauuaca agauu | 7 |
| aaucuuguaa uccugaaggc uccuc | 8 |
| gcugacccug aaguucaucu gcauu | 9 |
| aaugcagaug aacuucaggg ucagc | 10 |
| ggauccacga gcccaagggc uacca | 11 |
| ugguagcccu ugggcucgug gaucc | 12 |
| cccaagggcu accaugccaa cuucu | 13 |
| agaaguuggc augguagccc uuggg | 14 |
| gagcccaagg gcuaccaugc caacu | 15 |
| aguuggcaug guagcccuug ggcuc | 16 |
| gauccacgag cccaagggcu accau | 17 |
| augguagccc uugggcucgu ggauc | 18 |
| cacgagccca agggcuacca ugcca | 19 |
| uggcauggua gcccuugggc ucgug | 20 |
| gaggucaccc gcgugcuaau ggugg | 21 |
| ccaccauuag cacgcgggug accuc | 22 |
| guacaacagc acccgcgacc gggug | 23 |
| cacccggucg cgggugcugu uguac | 24 |
| guggauccac gagcccaagg gcuac | 25 |
| guagcccuug ggcucgugga uccac | 26 |
| ggucuggugc cuggucugau gaugu | 27 |
| acaucaucag accaggcacc agacc | 28 |
| gagcaccauu cuccuugaaa ggacu | 29 |
| aguccuuuca aggagaaugg ugcuc | 30 |
| ccucaauuca gucucucauc ugcaa | 31 |
| uugcagauga gagacugaau ugagg | 32 |
| gauguuugca uucuuugccc agcac | 33 |
| gugcugggca aagaaugcaa acauc | 34 |
| gucuuugguc uggugccugg ucuga | 35 |
| ucagaccagg caccagacca aagac | 36 |
| gugccugguc ugaugaugua ugcca | 37 |
| uggcauacau caucagacca ggcac | 38 |
| caccauucuc cuugaaagga cuuau | 39 |
| auaaguccuu ucaaggagaa uggug | 40 |
| caauucaguc ucucaucugc aauaa | 41 |
| uuauugcaga ugagagacug aauug | 42 |
| gguggcugga acagccagau guguu | 43 |
| aacacaucug gcuguuccag ccacc | 44 |
| gcuggaacag ccagaugugu ugcca | 45 |
| uggcaacaca ucuggcuguu ccagc | 46 |
| cgccagauua ccaucugguu ucaga | 47 |
| ucugaaacca gaugguaauc uggcg | 48 |
| ggagcccggc aauuaugcca ccuug | 49 |
| caagguggca uaauugccgg gcucc | 50 |
| caaggauauc gaaggcuugc uggga | 51 |
| ucccagcaag ccuucgauau ccuug | 52 |
| ggacaagagg cgcaagaucu cggca | 53 |
| ugccgagauc uugcgccucu ugucc | 54 |
| gcaagaucuc ggcagccacc agccu | 55 |
| aggcuggugg cugccgagau cuugc | 56 |
| ccaucugguu ucagaaccgc cgggu | 57 |
| acccggcggu ucugaaacca gaugg | 58 |

The following examples illustrate certain aspects of the disclosure and should not be construed as limiting the scope thereof.

### EXAMPLES

### Example 1

### Evaluation of STP707 Efficacy in Liver and Lung Tissue of Cynomolgus Monkeys Following Intravenous (IV) Administration

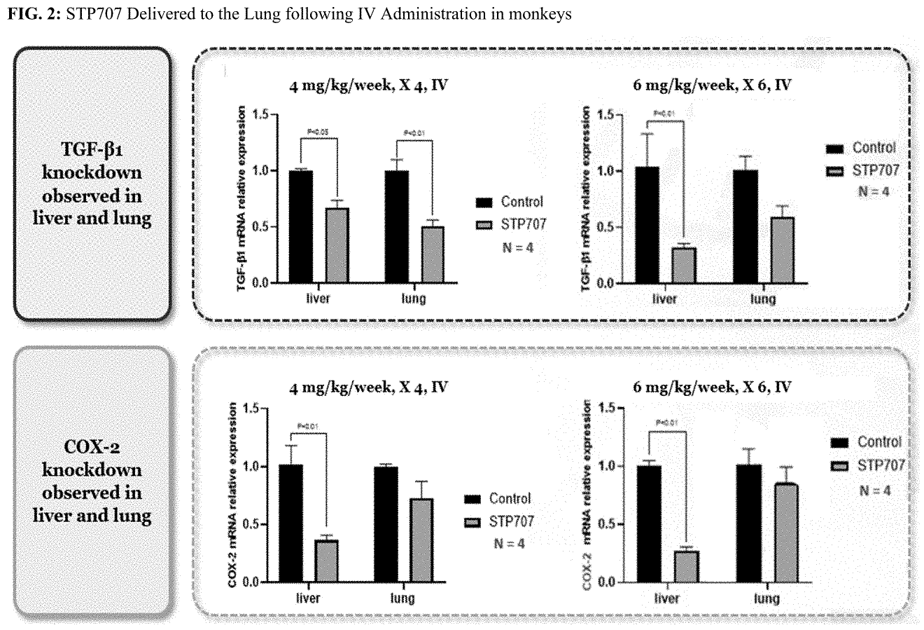

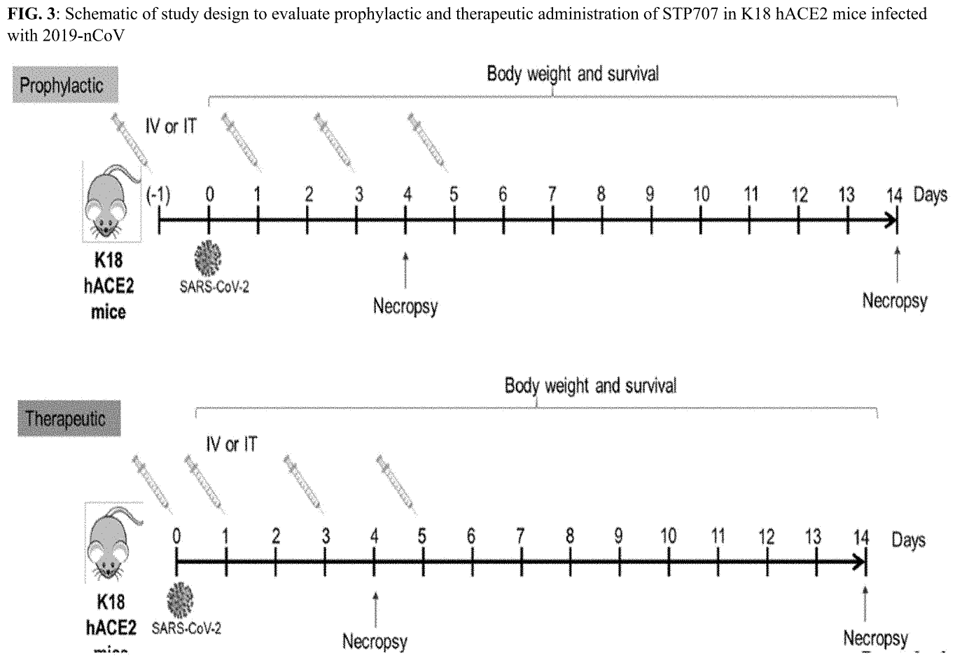

Methods: Sixteen Cynomolgus monkeys (Beijing Prima Biotech Inc.) were randomized and assigned to two control and two treatment groups (n=4 each) corresponding to either a four-week or a 6-week trial. Monkeys assigned to the four-week trial were administered intravenously 4 mg/kg each week; those assigned to the six-week trial were administered intravenously 6 mg/kg/each week. The animals were euthanized at the end of each trial, and lung and liver samples were analyzed for TGFB1 and Cox2 expression relative to RNA content.
Results: FIG. 2 shows results of the analysis. We observed significant reductions in expression of both TGFβ1 and Cox2 in liver and lung tissues of monkeys administered STP707 intravenously.
TGFβ1: In the 4-week trial the reductions were significant in both liver (p<0.5) and lung (p<0.1); at the 6-week trial only the reduction in liver was significant (p<0.1), although there appeared to be a trend toward a reduction in lung tissue. Nonetheless, this study demonstrated that IV administration of STP707 lead to delivery of the siRNA of lung tissue (at least against TGFβ1 gene expression).
Cox2: Significant reductions were seen in liver in both the four- and six-week trials (both p<0.1).

### Example 2

Evaluation of the Effect of Prophylactic and Therapeutic STP707 Administration Via Intravenous (IV) and Intratracheal (IT) Routes in k18 hACE2 Mice Infected with 2019-nCoV
Methods: Four to 6-week old k18 hACE2 female mice were randomized to 6 groups consisting of two control groups (PBS, mock infected, n=3; non-silencing (NS) siRNA (negative controls), n=10), and four treatments groups of 10 mice each receiving STP707 (2.5 mg/kg):
1. prophylactic IV administration of 40 μg on Day −1 & Days 1, 3, & 5 post infection;
2. prophylactic IT administration of 40 μg as with IV administration;
3. therapeutic IV administration of 40 μg on Days 0 (post infection) & Days 1, 3 & 5 post infection; and
4. therapeutic IT administration of 40 μg, as with IV administration.

All but the PBS control group were inoculated intranasally with 1×10³ PFU/animal of the 2019-nCoV. Four animals from each of the NS siRNA control and each treatment group were euthanized on Day 4 post infection and the following tissues were collected for analysis: (i) right caudal lung lobe (for histopathology if required); and (ii) remaining lung lobes and nasal turbinate (for homogenate preparation and determination of the viral load by plaque assay); the lung and nasal turbinate homogenate was also stored for cytokine determination (if required). All three PBS control group mice and the remaining 6 mice from each treatment group were monitored for morbidity and mortality until Day 14 post infection when they will be euthanized for the same tissue collection and analysis as indicated above. Any mice exhibiting labored breathing, eye discharge, nasal discharge, hunched posture and or substantial (20-25%) body weight loss were euthanized under IACUC instructions. Mice infected with the 2019-nCoV showed ˜60 percent survival by Day 14 post infection. FIG. 3 provides a schematic of the study design.

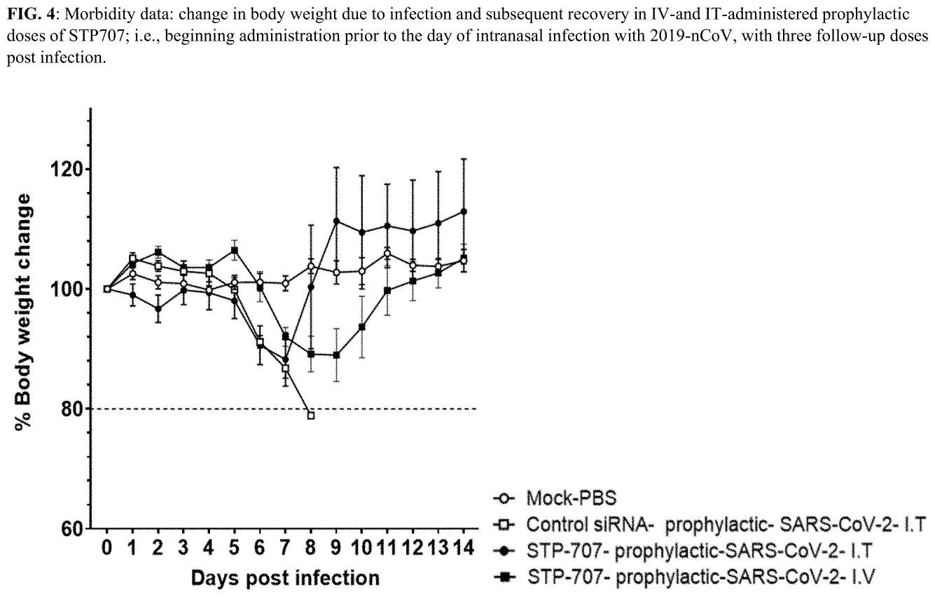

Results: FIGS. 4-8 provide morbidity and mortality data. FIG. 4 shows the initial loss and then gain of body weight among mice prophylactically administered STP707 through IV and IT routes, indicating that both routes of administration are efficacious as early as 7 to 10 days post infection. As indicated above, as mice in the control siRNA group continued to lose a significant amount of weight they were euthanized per the IACUC rules. FIGS. 5A-5D, likewise depict the change of body weight post infection among individual mice in the four groups. While the body weights of all of the mice in the IV administered group rebounded and the mice lived to Day 14, only 2 of 6 mice in the IT administered group lived to Day 14, suggesting a somewhat better response through prophylactic IV administration. FIG. 6 shows the initial loss and then gain of body weight among mice therapeutically administered STP707 through IV and IT routes. Without prophylactic administration through an IT route prior to the time of infection, the IT route was not a success. Only IV administration was shown to be effective. All mice therapeutically administered STP707 through the IT route died by Day 7. FIGS. 7A-7D depict the change of body weight post infection among individual mice in the four groups. As shown in FIGS. 7C and 7D, only one mouse survived beyond Day10, indicating that therapeutic administration of STP707, i.e., administration of STP707 on or after the date of exposure to the virus, may be only marginally successful, at least in this animal model. Finally, FIG. 8 shows mortality data for all control and treatment groups, indicating that all mice in the control groups had died by Day 7 post infection, whereas prophylactic delivery of STP707 (i.e., 24 hours in advance of infection) via the IV route resulted in 83 percent survival by Day 14.

### Example 3

### Evaluation of STP707 Efficacy in CD-1 Mouse Model

Methods. Fifteen female, 6-8-week-old CD-1 mice were assigned to one of three groups and acclimated for five days. On day 0, animals in each group were dosed intratracheally with one of (i) non-silencing siRNA with HKP+H, (ii) a single dose of STP707 (2 mg/Kg in 40 μL/animal) or (iii) two doses of STP707 (2×2 mg/Kg in 40 μL/animal). All animals were monitored for adverse effects. On day 3, the mice were euthanized and lung tissue was harvested to quantify TGFβ by real time quantitative reverse transcription PCR (qRT-PCR).

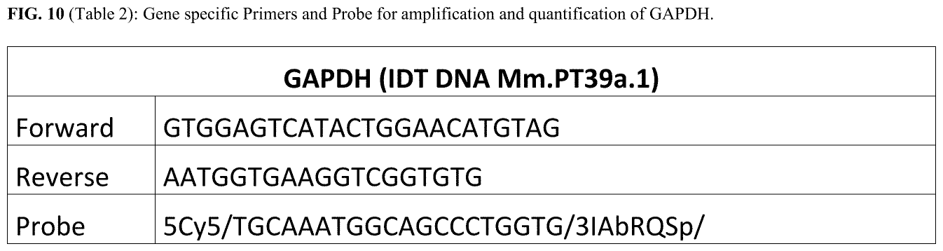

RNA concentration in each sample was determined using a nanodrop system. RNA was diluted to 100 ng/μL in molecular grade water and was used to program separate one-step qRT-PCR reactions for amplification of murine TGFβ (900 ng RNA) or murine GAPDH (250 ng RNA) using sequence-specific oligonucleotide primers and fluorescently-labeled oligonucleotide probes as shown in Table 1 (FIG. 9) and Table 2 (FIG. 10). Assays were performed in 96-well plates on an Applied Biosystems (Foster City, Calif.) QuantStudio 6. Data were collected by the Quant6 supporting software, and relative TGFβ expression levels were calculated using the ΔΔCt method, normalizing individual TGFβ expression levels to the GAPDH endogenous control; values were expressed relative to the siRNA control group. Data also were analyzed by normalizing TGFβ and GAPDH expression to the total siRNA used to program the reactions, and values were again expressed relative to the siRNA control group.

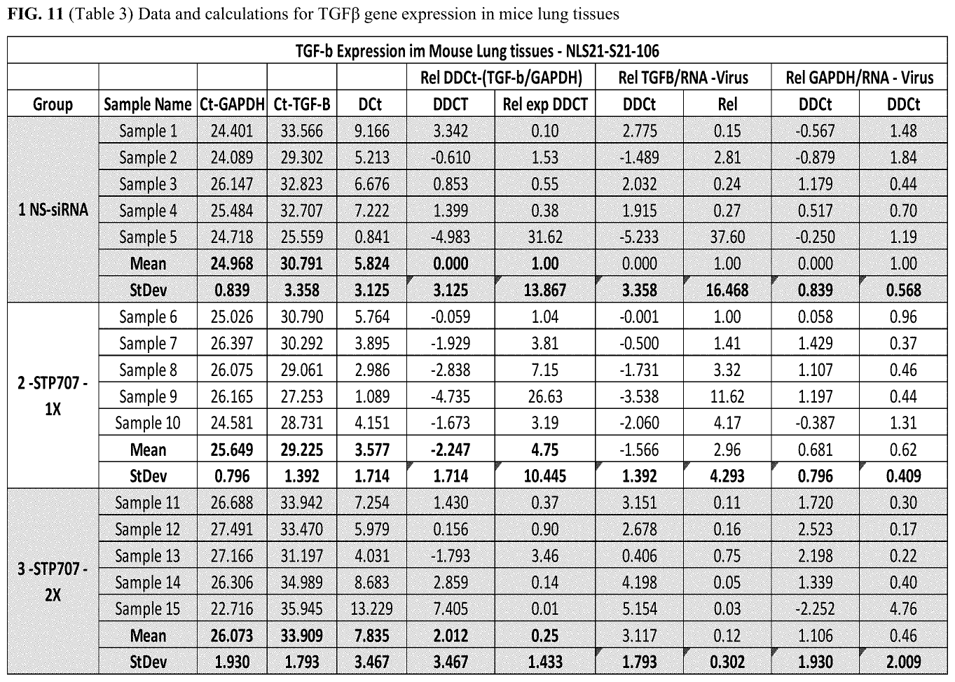

Results. Table 3 (FIG. 11) shows the experimental amplification data (Ct values) and the calculated dCt, DDCt and relative expression level for each sample. FIGS. 12A-C depict the effect of intratracheal administration of STP707 on TGFβ1 levels in the lung tissue of mice after (i) one dose; and (ii) two doses (mean±SD). The data demonstrate a dose-dependent reduction in TGFβ1 expression in lung tissue when STP707 is administered intratracheally, indicating that intratracheal administration of the pharmaceutical composition comprising HKP(+H) with TGFβ1 and Cox2 siRNAs is capable of entering lung tissue and silencing the targeted genes.

The disclosed embodiments described and claimed are not to be limited in scope by the specific preferred embodiments referenced herein, since these embodiments are intended as illustrations, not limitations. Any equivalent embodiments are intended to be within the scope of this disclosure, and the embodiments disclosed are not mutually exclusive. Indeed, various modifications to the embodiments, in addition to those shown and described herein will become apparent to those skilled in the art from the foregoing description. Such modifications are also intended to fall within the scope of the appended claims.

The terms and words used in the following description and claims are not limited to conventional definitions but, rather, are used to enable a clear and consistent understanding of the disclosure. Accordingly, it should be apparent to those skilled in the art that the description of various embodiments is provided for illustration purpose only and not for the purpose of limiting the disclosure with respect to the appended claims and their equivalents.

It is to be understood that the singular forms “a,” “an,” and “the” include the plural forms unless the context clearly dictates otherwise, e.g., reference to “a dermatologically active compound” includes reference to one or more such compounds.

Unless otherwise defined herein, all terms used have the same meaning as commonly understood by a person of ordinary skill in the art. Terms used herein should be interpreted as having meanings consistent with their meanings in the context of the relevant art.

As used herein, the terms “comprising,” “comprise” or “comprised,” in reference to defined or described elements of any item, composition, formulation, apparatus, method, process, system, etc., are intended to be inclusive or open ended, and includes those specified elements or their equivalents. Other elements can be included and still fall within the scope or definition of the defined item, composition, etc.

The term “about” or “approximately” means within an acceptable error range for the particular value as viewed by one of ordinary skill in the art; this depends in part on how the value is measured or determined based on the limitations of the measurement system.

“Co-administer” or “co-deliver” refers to the simultaneous administration of two pharmaceutical formulations in the blood or other fluid of an individual using the same or different modes of administration. Pharmaceutical formulations can be concurrently or sequentially administered in the same pharmaceutical carrier or in different ones.

The terms “subject,” “patient,” and “individual” are used interchangeably.

What is claimed is:

1. A method of preventing and/or ameliorating a 2019-nCoV infection comprising administering to a subject in need thereof a pharmaceutically effective amount of STP707.

2. The method of claim 1, wherein the STP707 is administered to said subject prior to exposure of the subject to 2019-nCoV.

3. The method of claim 1, wherein said administration of STP707 to a subject takes place at least 2 weeks, 10 days, 8 days, 6 days, 5 days, 4 days, 3 days, 2 days 1 day, 18 hours, 12 hours, 6 hours or 3 hours prior to exposure of the subject to the 2019-nCoV.

4. The method of claim 1, wherein said STP707 is administered intravenously.

5. The method of claim 1, wherein said STP707 is administered intratracheally.

6. A method of treating a subject suffering from, or suspected of suffering from, 2019-nCoV infection, comprising administering to the subject a pharmaceutically effective amount of STP707.

7. The method of claim 6, wherein said subject has not exhibited any known symptom of a 2019-nCoV infection.

8. The method of claim 6, wherein said STP707 is administered at least 3 hours, 6 hours, 12 hours, 18 hours, 1 day, 2 days, 4 days, 6 days, 8 days, 10 days, 2 weeks, 3 weeks, 4 weeks, 6 weeks or 8 weeks following exposure of the subject to the 2019 nCoV.

9. The method of claim 6, wherein said STP707 is administered intravenously.

10. The method of claim 6, wherein said STP707 is administered intratracheally.

11. The method of claim 1, where said subject exhibits an elevated serum concentration of TGFβ1 compared to that in a standard range for healthy subjects.

12. The method of claim 11 wherein the concentration of TGFβ1 is determined through an enzyme-linked immunosorbent assay (ELISA).

13. The method of claim 11, wherein said subject is diagnosed with a 2019-nCoV infection and is hospitalized, and wherein the subject exhibits an elevated TGFβ1 concentration in blood at any time between an admittance day and 21 days following the admittance day.

14. The method of treating the subject of claim 6, wherein said STP707 is administered to said subject initially within 1 day, 2 days, 3 days, or 4 days following initial determination of said elevated TGFβ1 concentration in said subject's blood.

15. The method of claim 14 wherein the subject continues to be treated by administration of STP707 following said initial administration.

16. The method of claim 11, wherein said STP707 is administered intravenously.

17. The method of claim 11, wherein said STP707 is administered intratracheally.

18. A method of slowing the progression of a 2019-nCoV infection in a subject comprising administering to the subject a pharmaceutically effective amount of STP707.

19. The method of subject of claim 18, wherein said STP707 is administered within 3 hours, 6 hours, 12 hours, 18 hours, 1 day, 2 days, 4 days, 6 days, 8 days, 10 days, 2 weeks, 3 weeks, 4 weeks, 6 weeks or 8 weeks following exposure of the subject to the 2019-nCoV.

20. The method of claim 18, wherein said STP707 is administered intravenously.

21. The method of claim 18, wherein said STP707 is administered intratracheally.

22. The method of claim 1, wherein the subject is a mammal, and is selected from the group consisting of humans, non-human primates, mice, rats and ferrets.

23. The method of claim 22, wherein the subject is a human.


SEQUENCE LISTING (58 sequences)

SEQ ID NO: 1 - RNA, 25 nt, synthetic construct
  misc_feature 1..25: note = synthetic
  source 1..25: mol_type = other RNA
cccaagggctaccatgccaacttct

SEQ ID NO: 2 - RNA, 25 nt, synthetic construct
  misc_feature 1..25: note = synthetic
  source 1..25: mol_type = other RNA
agaagttggcatggtagcccttggg

SEQ ID NO: 3 - RNA, 25 nt, synthetic construct
  misc_feature 1..25: note = synthetic
  source 1..25: mol_type = other RNA
ggtctggtgcctggtctgatgatgt

SEQ ID NO: 4 - RNA, 25 nt, synthetic construct
  misc_feature 1..25: note = synthetic
  source 1..25: mol_type = other RNA
acatcatcagaccaggcaccagacc

SEQ ID NO: 5 - RNA, 25 nt, synthetic construct
  misc_feature 1..25: note = synthetic
  source 1..25: mol_type = other RNA
ggtggctggaacagccagatgtgtt

SEQ ID NO: 6 - RNA, 25 nt, synthetic construct
  misc_feature 1..25: note = synthetic
  source 1..25: mol_type = other RNA
aacacatctggctgttccagccacc

SEQ ID NO: 7 - RNA, 25 nt, synthetic construct
  misc_feature 1..25: note = synthetic
  source 1..25: mol_type = other RNA
gaggagccttcaggattacaagatt

SEQ ID NO: 8 - RNA, 25 nt, synthetic construct
  misc_feature 1..25: note = synthetic
  source 1..25: mol_type = other RNA
aatcttgtaatcctgaaggctcctc

SEQ ID NO: 9 - RNA, 25 nt, synthetic construct
  misc_feature 1..25: note = synthetic
  source 1..25: mol_type = other RNA
gctgaccctgaagttcatctgcatt

SEQ ID NO: 10 - RNA, 25 nt, synthetic construct
  misc_feature 1..25: note = synthetic
  source 1..25: mol_type = other RNA
aatgcagatgaacttcagggtcagc

SEQ ID NO: 11 - RNA, 25 nt, synthetic construct
  misc_feature 1..25: note = synthetic
  source 1..25: mol_type = other RNA
ggatccacgagcccaagggctacca

SEQ ID NO: 12 - RNA, 25 nt, synthetic construct
  misc_feature 1..25: note = synthetic
  source 1..25: mol_type = other RNA
tggtagcccttgggctcgtggatcc

SEQ ID NO: 13 - RNA, 25 nt, synthetic construct
  misc_feature 1..25: note = synthetic
  source 1..25: mol_type = other RNA
cccaagggctaccatgccaacttct

SEQ ID NO: 14 - RNA, 25 nt, synthetic construct
  misc_feature 1..25: note = synthetic
  source 1..25: mol_type = other RNA
agaagttggcatggtagcccttggg

SEQ ID NO: 15 - RNA, 25 nt, synthetic construct
  misc_feature 1..25: note = synthetic
  source 1..25: mol_type = other RNA
gagcccaagggctaccatgccaact

SEQ ID NO: 16 - RNA, 25 nt, synthetic construct
  misc_feature 1..25: note = synthetic
  source 1..25: mol_type = other RNA
agttggcatggtagcccttgggctc

SEQ ID NO: 17 - RNA, 25 nt, synthetic construct
  misc_feature 1..25: note = synthetic
  source 1..25: mol_type = other RNA
gatccacgagcccaagggctaccat

SEQ ID NO: 18 - RNA, 25 nt, synthetic construct
  misc_feature 1..25: note = synthetic
  source 1..25: mol_type = other RNA
atggtagcccttgggctcgtggatc

SEQ ID NO: 19 - RNA, 25 nt, synthetic construct
  misc_feature 1..25: note = synthetic
  source 1..25: mol_type = other RNA
cacgagcccaagggctaccatgcca

SEQ ID NO: 20 - RNA, 25 nt, synthetic construct
  misc_feature 1..25: note = synthetic
  source 1..25: mol_type = other RNA
tggcatggtagcccttgggctcgtg

SEQ ID NO: 21 - RNA, 25 nt, synthetic construct
  misc_feature 1..25: note = synthetic
  source 1..25: mol_type = other RNA
gaggtcacccgcgtgctaatggtgg

SEQ ID NO: 22 - RNA, 25 nt, synthetic construct
  misc_feature 1..25: note = synthetic
  source 1..25: mol_type = other RNA
ccaccattagcacgcgggtgacctc

SEQ ID NO: 23 - RNA, 25 nt, synthetic construct
  misc_feature 1..25: note = synthetic
  source 1..25: mol_type = other RNA
gtacaacagcacccgcgaccgggtg

SEQ ID NO: 24 - RNA, 25 nt, synthetic construct
  misc_feature 1..25: note = synthetic
  source 1..25: mol_type = other RNA
cacccggtcgcgggtgctgttgtac

SEQ ID NO: 25 - RNA, 25 nt, synthetic construct
  misc_feature 1..25: note = synthetic
  source 1..25: mol_type = other RNA
gtggatccacgagcccaagggctac

SEQ ID NO: 26 - RNA, 25 nt, synthetic construct
  misc_feature 1..25: note = synthetic
  source 1..25: mol_type = other RNA
gtagcccttgggctcgtggatccac

SEQ ID NO: 27 - RNA, 25 nt, synthetic construct
  misc_feature 1..25: note = synthetic
  source 1..25: mol_type = other RNA
ggtctggtgcctggtctgatgatgt

SEQ ID NO: 28 - RNA, 25 nt, synthetic construct
  misc_feature 1..25: note = synthetic
  source 1..25: mol_type = other RNA
acatcatcagaccaggcaccagacc

SEQ ID NO: 29 - RNA, 25 nt, synthetic construct
  misc_feature 1..25: note = synthetic
  source 1..25: mol_type = other RNA
gagcaccattctccttgaaaggact

SEQ ID NO: 30 - RNA, 25 nt, synthetic construct
  misc_feature 1..25: note = synthetic
  source 1..25: mol_type = other RNA
agtcctttcaaggagaatggtgctc

SEQ ID NO: 31 - RNA, 25 nt, synthetic construct
  misc_feature 1..25: note = synthetic
  source 1..25: mol_type = other RNA
cctcaattcagtctctcatctgcaa

SEQ ID NO: 32 - RNA, 25 nt, synthetic construct
  misc_feature 1..25: note = synthetic
  source 1..25: mol_type = other RNA
ttgcagatgagagactgaattgagg

SEQ ID NO: 33 - RNA, 25 nt, synthetic construct
  misc_feature 1..25: note = synthetic
  source 1..25: mol_type = other RNA
gatgtttgcattctttgcccagcac

SEQ ID NO: 34 - RNA, 25 nt, synthetic construct
  misc_feature 1..25: note = synthetic
  source 1..25: mol_type = other RNA
gtgctgggcaaagaatgcaaacatc

SEQ ID NO: 35 - RNA, 25 nt, synthetic construct
  misc_feature 1..25: note = synthetic
  source 1..25: mol_type = other RNA
gtctttggtctggtgcctggtctga

SEQ ID NO: 36 - RNA, 25 nt, synthetic construct
  misc_feature 1..25: note = synthetic
  source 1..25: mol_type = other RNA
tcagaccaggcaccagaccaaagac

SEQ ID NO: 37 - RNA, 25 nt, synthetic construct
  misc_feature 1..25: note = synthetic
  source 1..25: mol_type = other RNA
gtgcctggtctgatgatgtatgcca

SEQ ID NO: 38 - RNA, 25 nt, synthetic construct
  misc_feature 1..25: note = synthetic
  source 1..25: mol_type = other RNA
tggcatacatcatcagaccaggcac

SEQ ID NO: 39 - RNA, 25 nt, synthetic construct
  misc_feature 1..25: note = synthetic
  source 1..25: mol_type = other RNA
caccattctccttgaaaggacttat

SEQ ID NO: 40 - RNA, 25 nt, synthetic construct
  misc_feature 1..25: note = synthetic
  source 1..25: mol_type = other RNA
ataagtcctttcaaggagaatggtg

SEQ ID NO: 41 - RNA, 25 nt, synthetic construct
  misc_feature 1..25: note = synthetic
  source 1..25: mol_type = other RNA
caattcagtctctcatctgcaataa

SEQ ID NO: 42 - RNA, 25 nt, synthetic construct
  misc_feature 1..25: note = synthetic
  source 1..25: mol_type = other RNA
ttattgcagatgagagactgaattg

SEQ ID NO: 43 - RNA, 25 nt, synthetic construct
  misc_feature 1..25: note = synthetic
  source 1..25: mol_type = other RNA
ggtggctggaacagccagatgtgtt

SEQ ID NO: 44 - RNA, 25 nt, synthetic construct
  misc_feature 1..25: note = synthetic
  source 1..25: mol_type = other RNA
aacacatctggctgttccagccacc

SEQ ID NO: 45 - RNA, 25 nt, synthetic construct
  misc_feature 1..25: note = synthetic
  source 1..25: mol_type = other RNA
gctggaacagccagatgtgttgcca

SEQ ID NO: 46 - RNA, 25 nt, synthetic construct
  misc_feature 1..25: note = synthetic
  source 1..25: mol_type = other RNA
tggcaacacatctggctgttccagc

SEQ ID NO: 47 - RNA, 25 nt, synthetic construct
  misc_feature 1..25: note = synthetic
  source 1..25: mol_type = other RNA
cgccagattaccatctggtttcaga

SEQ ID NO: 48 - RNA, 25 nt, synthetic construct
  misc_feature 1..25: note = synthetic
  source 1..25: mol_type = other RNA
tctgaaaccagatggtaatctggcg

SEQ ID NO: 49 - RNA, 25 nt, synthetic construct
  misc_feature 1..25: note = synthetic
  source 1..25: mol_type = other RNA
ggagcccggcaattatgccaccttg

SEQ ID NO: 50 - RNA, 25 nt, synthetic construct
  misc_feature 1..25: note = synthetic
  source 1..25: mol_type = other RNA
caaggtggcataattgccgggctcc

SEQ ID NO: 51 - RNA, 25 nt, synthetic construct
  misc_feature 1..25: note = synthetic
  source 1..25: mol_type = other RNA
caaggatatcgaaggcttgctggga

SEQ ID NO: 52 - RNA, 25 nt, synthetic construct
  misc_feature 1..25: note = synthetic
  source 1..25: mol_type = other RNA
tcccagcaagccttcgatatccttg

SEQ ID NO: 53 - RNA, 25 nt, synthetic construct
  misc_feature 1..25: note = synthetic
  source 1..25: mol_type = other RNA
ggacaagaggcgcaagatctcggca

SEQ ID NO: 54 - RNA, 25 nt, synthetic construct
  misc_feature 1..25: note = synthetic
  source 1..25: mol_type = other RNA
tgccgagatcttgcgcctcttgtcc

SEQ ID NO: 55 - RNA, 25 nt, synthetic construct
  misc_feature 1..25: note = synthetic
  source 1..25: mol_type = other RNA
gcaagatctcggcagccaccagcct

SEQ ID NO: 56 - RNA, 25 nt, synthetic construct
  misc_feature 1..25: note = synthetic
  source 1..25: mol_type = other RNA
aggctggtggctgccgagatcttgc

SEQ ID NO: 57 - RNA, 25 nt, synthetic construct
  misc_feature 1..25: note = synthetic
  source 1..25: mol_type = other RNA
ccatctggtttcagaaccgccgggt

SEQ ID NO: 58 - RNA, 25 nt, synthetic construct
  misc_feature 1..25: note = synthetic
  source 1..25: mol_type = other RNA
acccggcggttctgaaaccagatgg